(19) United States
(12) Patent Application Publication
(10) Pub. No.: US 2026/0002133 A1
(43) Pub. Date: Jan. 1, 2026

(54) 3D Bioprinted Tissues and Screening Assays Therewith

(71) Applicant: The Government of the United States, as Represented by the Director of the Defense Health Agency, Fort Detrick, MD (US)

(72) Inventors: Yantenew Gete, Silver Spring, MD (US); Eric Huy-Dang Nguyen, Clarksville, MD (US); Rajini Reddy Mudhasani, Frederick, MD (US)

(21) Appl. No.: 19/237,101

(22) Filed: Jun. 13, 2025

Related U.S. Application Data
(60) Provisional application No. 63/665,480, filed on Jun. 28, 2024.

(51) Int. Cl. C12N 5/071; B29C 64/112; B29L 31/40; B33Y 10/00; B33Y 70/10; B33Y 80/00

(52) U.S. Cl. CPC C12N 5/069; B29C 64/112; B33Y 10/00; B33Y 70/10; B33Y 80/00; C12N 5/0691; B29K 2089/00; B29L 2031/40; C12N 2503/04; C12N 2513/00; C12N 2533/54; C12N 2533/90

(57) ABSTRACT

Disclosed herein are 3D printed vascular tissues and compositions and methods for making the 3D printed vascular tissues. The 3D printed vascular tissues can be used to model vascular tissue damage (e.g., vascular leakage) and in screening assays for agents that cause or inhibit or treat vascular tissue damage.

### CROSS-REFERENCE TO RELATED APPLICATIONS

This application claims the benefit of U.S. Patent Application No. 63/665,480, filed Jun. 28, 2025, which is herein incorporated by reference in its entirety.

This invention was made with Government support from the United States Army, Military Infectious Diseases Research Program. The United States Government has certain rights in the invention.

### BACKGROUND OF THE INVENTION

### 1. Field of the Invention

The field of the invention generally relates to 3D printed tissues and methods of using such to characterize and/or screen viral hemorrhagic fever pathogenesis.

### 2. Description of the Related Art

Endothelial cells (EC) line up to form a single layer of cells in the inner most lining of a blood vessel wall and play a role in the regulation of vascular functions. Under quiescent conditions, ECs retain the fluid within the blood vessel while promoting the exchange of gasses and certain metabolites in between the blood or lymph and the surrounding tissues. The fluid retention is achieved by the tight linking of cells to each other and by the smooth coating of a proteoglycan layer also known as endothelial glycocalyx layer (EGL) present on the luminal surface of the endothelium. The tight links between the cells are established by different types of adhesive structures and cell to cell junctions. Adherens junctions (AJ), formed by vascular endothelial (VE) cadherins, are the major type of cell-to-cell junction in endothelium and prevent fluid loss from the blood vessel. This barrier function is further enhanced by EGL, which is composed of glycosylated proteins, sugars, and lipids. The negative charge on the EGL prevents the non-specific binding of blood cells to the endothelium and promotes the smooth passage of blood cells through the lumen. The coating also provides the necessary strength to endure the mechanical forces of the bloodstream. Under quiescent conditions, the endothelium regulates the homeostasis of peripheral tissues. Most important, EC also responds to a myriad of systemic and locally generated factors that alter the fluid barrier properties in response to the changing milieu in the surrounding tissue as described below.

Upon viral infection, the ECs are activated by viral factors or by locally produced cytokines. Such activation promotes antiviral responses, recruitment, and translocation of leukocytes to the site of infection, clotting and vascular permeability. These responses collectively promote host responses to clear the infection. At the cellular level, the activated ECs cause the breakdown of EGL, express cell attachment factors known as leukocyte attachment factors (LAFs), which allow binding of circulating immune cells, such as monocytes, NK cells, T cells, and B cells, to the endothelium and promote their translocation across the blood vessel barrier to the site of infection. Normally, these responses are controlled by multiple intertwined networks of signaling cascades that restore the endothelium to a resting state to prevent fluid loss and clotting. However, prolonged EC activation by certain viruses lead to excessive accumulation of fluids in the tissues, edema, hypovolemic shock, multiorgan failure and clotting, a condition that is described by the term viral hemorrhagic fever (VHF).

Many viruses infect the endothelium but only a few disrupt EC and vascular function leading to VHF. Currently, VHFs represent a group of diseases caused by RNA viruses belonging to 6 taxonomy families: Filoviruses, Arenaviruses, Flaviviruses, Hantaviruses, Nairoviruses, and Phenuiviruses. Crimean-Congo hemorrhagic fever virus (CCHFV) and Dengue virus (DENV) belonging to the Nairoviridae and Flaviviridae family respectively are two such viruses that cause a mild to severe VHF by disruption of endothelium function.

DENV is the most prevalent arthropod borne virus and is a serocomplex of four viruses (DENV-1 to 4). Every year, 40% of the world population and about 129 countries in the world are at risk of Dengue viral (DENV) infections. The mechanism of vascular leakage during DENV infections is poorly understood at microvascular level. DENV, which is partly due to the absence of any suitable ex vivo model that recapitulates the in vivo defects. Thus, there is an urgent need to develop a suitable ex vivo model that closely recapitulates the in vivo vascular structure and preferably in a robust and high throughput manner.

### SUMMARY OF THE INVENTION

In some embodiments, the present invention provides a bioink composition comprising or consisting essentially of aortic smooth muscle cells (SMCs) and microvascular endothelial cells (MVECs) in an extracellular matrix solution which is a mixture of a gelatin solution, a basement membrane extract solution, and an ECL cell attachment matrix solution. In some embodiments, the gelatin solution is type A porcine skin gelatin at a concentration of 60 mg/mL; the basement membrane extract solution has a protein concentration of 15 mg/mL; the ECL cell attachment matrix solution has a protein concentration of 1 mg/mL; and wherein the extracellular matrix solution comprises the gelatin solution, the basement membrane extract solution, and the ECL cell attachment matrix solution in a ratio of 1:0.5:0.5 v/v. In some embodiments, the concentration of the aortic smooth muscle cells (SMCs) is about 0.5×10⁶ to about 1.5×10⁶ cells/mL, preferably about 1×10⁶ cells/mL, of the extracellular matrix solution and the concentration of the microvascular endothelial cells (MVECs) is about 0.5×10⁶ to about 1.5×10⁶ cells/mL, preferably about 1×10⁶ cells/mL, of the extracellular matrix solution. In some embodiments, the basement membrane extract solution is lactose dehydrogenase elevating virus (LDEV) free. In some embodiments, the basement membrane extract solution is LDEV free Gibco™ Geltrex™ available from Thermo Fisher Scientific.

In some embodiments, the present invention is directed to a method of making a 3D printed tissue, which comprises using a bioprinter having a 22 gauge nozzle, 10-12 kPa printing pressure, a 3 mm/s travel speed, and a 12° C. printbed temperature to print a bioink composition as disclosed herein in a grid infill pattern at a 20% grid density. In some embodiments, the density of the 3D printed tissue is equivalent to about 1 ml of bioink per about 1.25 cm³ of printed tissue. In some embodiments, the method comprises printing SMCs and MVECs at a concentration of about 8×10⁵ SMCs and about 8×10⁵ MVECs per about 1 cm³ of printed tissue. In some embodiments, the method further comprises culturing the 3D printed tissue in a cell culture medium for up to about 5 days at 37° C. In some embodiments, further comprises culturing the 3D printed tissue in a cell culture medium for 5 days at 37° C.

In some embodiments, the present invention is directed to a 3D printed tissue made by the methods disclosed herein. In some embodiments, the 3D printed tissues comprise at least about 8×10⁵ SMCs per about 1 cm³ of printed tissue and/or at least about 8×10⁵ MVECs per about 1 cm³ of printed tissue. In some embodiments, about 1 cm³ of the 3D printed tissue comprises at least about 8×10⁵ SMCs and at least about 8×10⁵ MVECs. In some embodiments, 1 cm³ of the 3D printed tissues comprise about 8×10⁵ SMCs and about 8×10⁵ MVECs after printing and before tissue maturation.

In some embodiments, the present invention is directed to a kit comprising a bioink composition as disclosed herein and/or the 3D printed tissue as disclosed herein packaged together with one or more reagents (e.g., buffers, growth media, cell culture media, detectable labels, etc.).

In some embodiments, the present invention is directed to a method of assaying whether an agent likely changes the structure of vascular tissue, which comprises contacting the agent with a 3D printed tissue as disclosed herein and then identifying any change in a cell structure in the 3D printed tissue, wherein a change in the cell structure in the 3D printed tissue indicates that the agent likely changes the structure of vascular tissue. In some embodiments, the change in the cell structure is identified by comparing the cell structure in the 3D printed tissue which was contacted with the agent with a control. In some embodiments, the control is a negative control (e.g., a 3D printed tissue that has not been contacted with the agent), a positive control (e.g., a 3D printed tissue that has been contacted with a given agent that is known to change the structure of vascular tissue), or a reference value (e.g., number of αSMA-positive cells present in a negative 3D printed control sample). In some embodiments, the agent is a virus, preferably a virus that causes viral hemorrhagic fever.

In some embodiments, the present invention is directed to a method of assaying whether a test agent likely inhibits or treats vascular tissue damage, which comprises contacting the 3D printed tissue as disclosed herein with (a) the test agent, and (b) an agent that is known to cause vascular tissue damage; and then identifying any change in a cell structure in the 3D printed tissue compared to a control, wherein the absence of a change in the cell structure in the 3D printed tissue indicates that the test agent likely inhibits or treats vascular tissue damage. In some embodiments, the test agent is contacted with the 3D printed tissue before, concurrently with, or after contact with the agent. In some embodiments, the agent is a virus, preferably a virus that causes viral hemorrhagic fever. In some embodiments, the test agent is Zanamivir, Cathepsin-L, or Rock Inhibitor Y-27632.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

The patent or application file contains at least one drawing executed in color. Copies of this patent or patent application publication with color drawing(s) will be provided by the Office upon request and payment of the necessary fee. This invention is further understood by reference to the drawings wherein:

FIG. 1: Workflow of 3D bioprinting of vascular tissue. Bioprinting process starts with preparing the bioink (a mixture of endothelial, smooth muscle cells, and an extracellular matrix), printing the desired cell-laden scaffold, and apply the matured 3D bioprinted vascular tissue for drug testing or in vitro disease modeling.

FIG. 2 to FIG. 6: Dengue Virus infection of human Aortic Smooth Muscle Cells. FIG. 2, FIG. 3: Aortic SMCs were used as infected cell types during optimization of DENV across 4 strains and 0-1 MOI. FIG. 2 shows the SMC cell count and FIG. 3 shows the % virus positive. FIG. 4: Representative Immunofluorescent images of human aortic SMCs infected with DENV-2 for 72 hours with 0.5 MOI, and fixed cells were probed with DENV antibody (green), CellMask cytoplasm stain (red), Hoechst nuclear stain (blue). FIG. 5, FIG. 6: 57% of the infected cells died after the infection, and the surviving cells were 56% positive for DENV infection. FIG. 5 shows the SMC cell count and FIG. 6 shows the % virus positive. *=p<0.05; ****=p<0.0001, one-way ANOVA and Dunnett's Multiple Comparisons Test. Scale bar: 200 μm.

FIG. 7 and FIG. 8: 3D bioprinted vascular tissue using microvascular lung endothelial cells (MVLECs) and human aortic smooth muscle cells (SMAs) after five days of maturation. FIG. 7: Although the bioink (MVLECs and SMC with extracellular matrix) mixed as a homogenous solution, between 5 days of maturation, cells organize such that SMA+ cells line the edges of the tissues and form a vascular wall. Red: SMA. Green: VE-Cadherin. Blue: Nuclei. FIG. 8: Image processing and quantification of DENV infection. Following five days of maturation, 3D printed vascular tissue models were infected with DENV strain 2 at 0.5 MOI for 72 hours. SMA+ cell number were significantly decreased with DENV infection. *=p<0.05, Student's T-Test. Each replicate represents a separate location on a printed tissue.

FIG. 9: Dengue Virus infection of 3D-Bioprinted vascular tissue models. Upon infection with DENV (MOI 0.5, 72 hours), cells on the edges of the MLBW constructs begin staining positive for DENV viral antigen (green); blue: Nuclei. Scale bar: 100 μm.

FIG. 10 to FIG. 13: Drug intervention of DENV infected 3D-Bioprinted vascular tissue. FIG. 10: Representative immunofluorescent images of uninfected, DENV infected, Zanamivir, Cathespin-L inhibitor, and ROCK inhibitor treated 3D bioprinted vascular tissue. Following 5 days of maturation, 3D printed vascular tissue were infected with Dengue Virus Strain 2 at 0.5 MOI for 72 hours. The main impact of DENV infection was the reduction of overall cell number and SMA+ cell number. FIG. 11, FIG. 12, FIG. 13: Treatment with 100 μM Zanamivir, 20 μM Cathepsin-L Inhibitor or 10 μM ROCK Inhibitor alleviated DENV-driven smooth muscle cell loss (FIG. 11), while Zanamivir and ROCK Inhibitor alleviated significant SMA+ cell loss (FIG. 12). No significant changes to VE-Cadherin+ cell properties were observed with DENV infection or drug treatment (FIG. 13). Red: SMA; Green: VE-Cadherin; Blue: Nuclei. *, p<0.05; **, p<0.01; ***, p<0.001; ****, p<0.0001, one-way ANOVA followed by Dunnett's Multiple Comparisons Test against DENV treatment. Each replicate represents a separate location on a printed tissue.

FIG. 14: Schematically shows the differentiation process for Example 3.

FIG. 15: Pictures of stained populations of iPSC-SMCs that expressed α-smooth muscle actin (α-SMA) and calponin.

FIG. 16: Pictures of stained populations of iPSC-ECs that expressed human vascular endothelial cadherin (hVE-Cadherin) and platelet endothelial cell adhesion molecule-1 (PECAM-1, also known as CD31).

FIG. 17: Pictures showing that IPSC-SMCs demonstrate contractile function and significant reduction in cell surface area following treatment using U46619, a vasoconstricting drug operating as a thromboxane A2 receptor agonist.

FIG. 18: Picture showing that iPSC-ECs form capillary-like microvascular networks in a tube formation assay.

FIG. 19: Picture demonstrating uptake of Alexa 488-labeled acetylated Low-Density Lipoproteins (LDL) by iPSC-ECs.

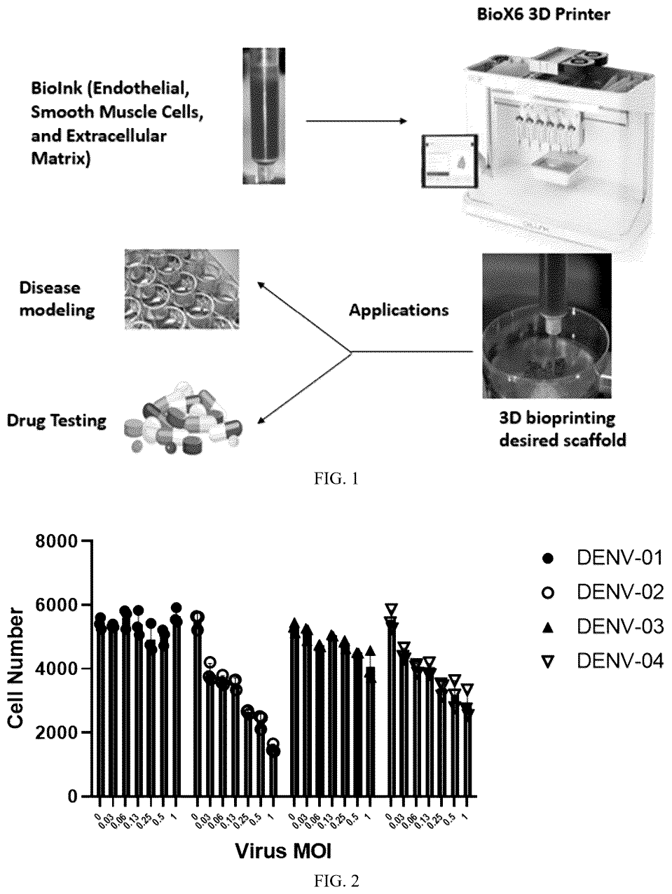

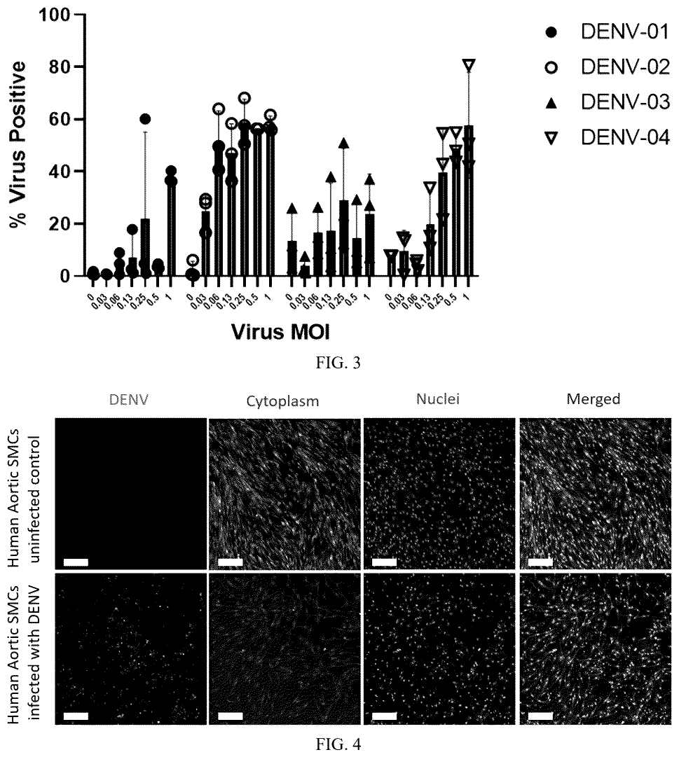

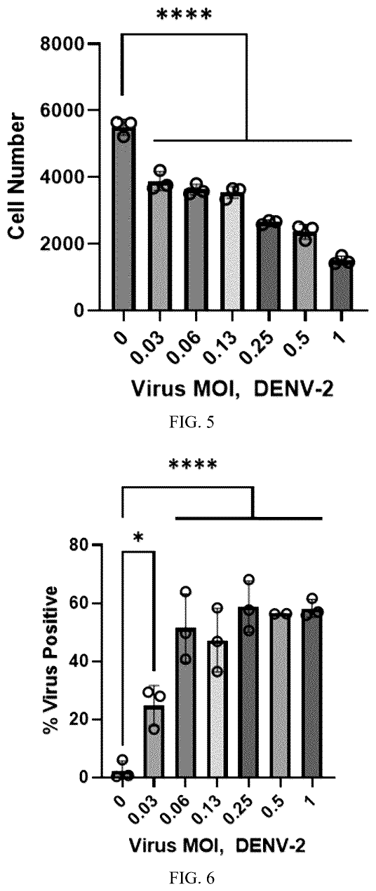

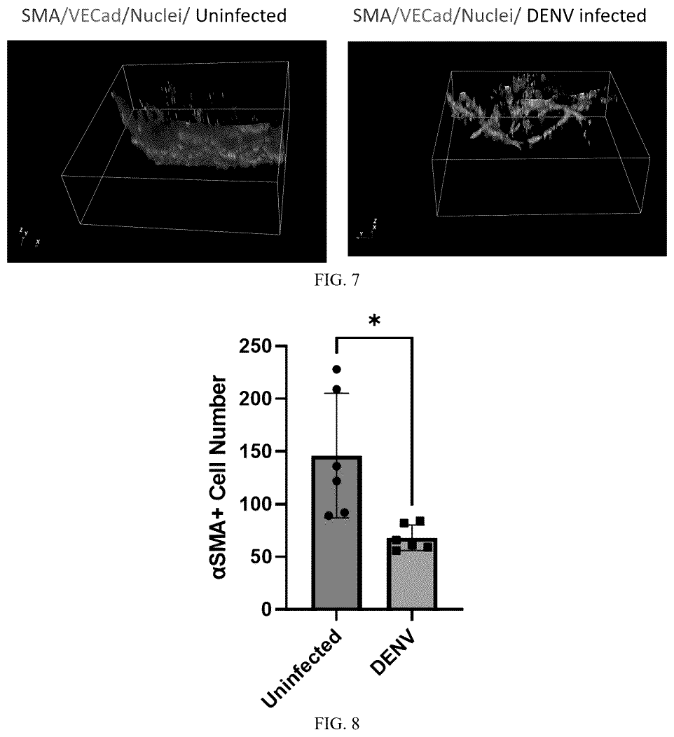

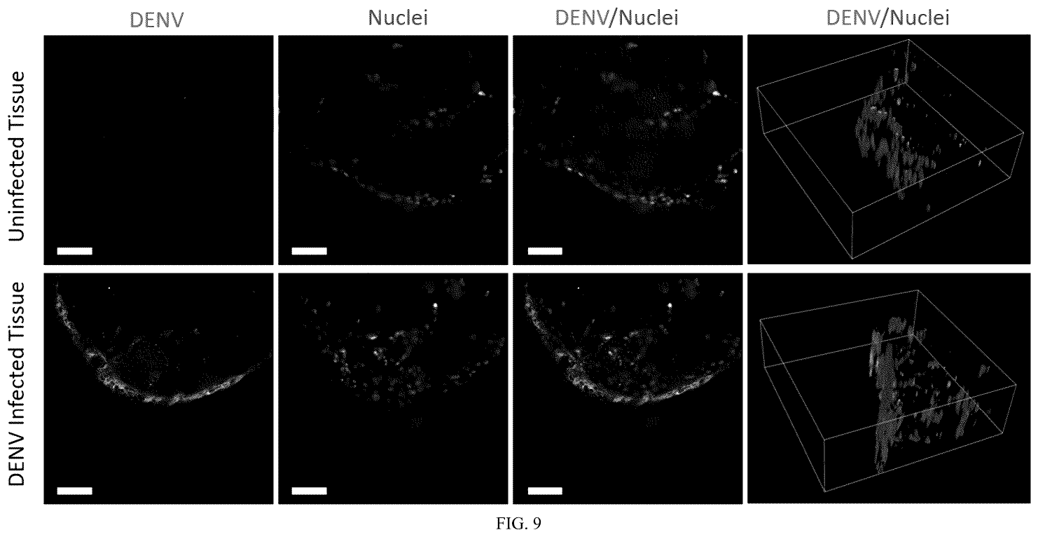

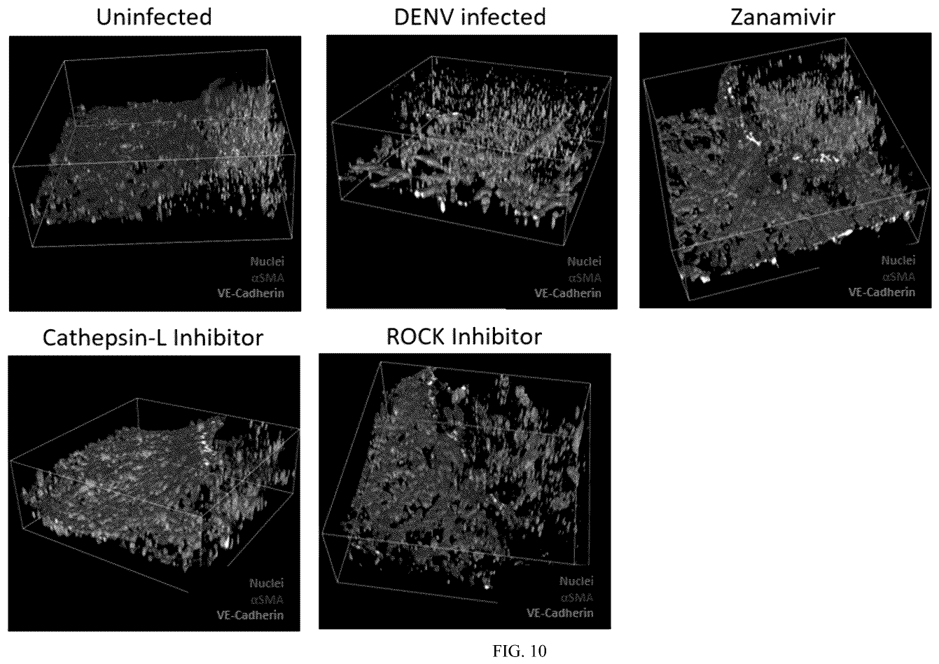

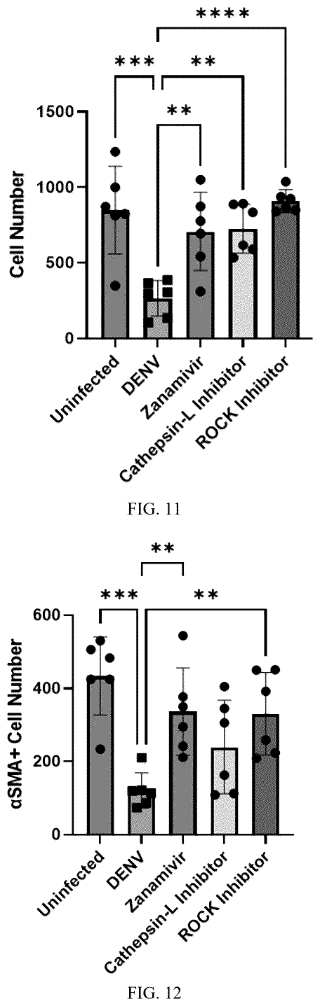

### DETAILED DESCRIPTION OF THE INVENTION

3D bioprinting uses cells and biomaterials as bio-ink which can precisely control the spatial layout of cells and surrounding microenvironment to recapitulate the native in vivo environment. This environment can be used to build in vitro tissue or organ models with great potential for application in drug screening and disease modeling. Although 2D cell culture and animal models are commonly used for new drug development and testing, 2D cell culture lacks the physiological 3D environment, and animal models are expensive, time-consuming, and differ from human metabolism leading to greater candidate drug failure rates. These highlight the need for developing cost-effective models that simulate the human physiological environment.

Disclosed herein are 3D printed tissues, bioinks for making the 3D printed tissues, and methods of making and using thereof. As disclosed herein, 3D bioprinted vascular tissue was created and used to model viral hemorrhagic fever (VHF) pathogenesis and screen for compounds that treat or inhibit VHF and/or pathogenesis. As disclosed herein dengue virus (DENV) was employed as the representative species of viruses that cause VHF.

The 3D bioprinted vascular tissue models the vascular leakage seen with VHF pathogenesis such as that resulting from infection by DENV. During a 5-day maturation period, smooth muscle cells migrated to the exterior of the construct and formed what resembled a smooth muscle wall which commonly envelops large blood vessels. DENV infection caused a loss of smooth muscle actin compared to negative controls. Treatment with Zanamivir, Cathepsin-L, or Rock Inhibitor Y-27632 inhibited the loss of smooth muscle actin caused by DENV infection. That is, the 3D bioprinted vascular tissues may be used to model vascular damage caused by VHF and screen candidate compounds for those that treat or inhibit vascular damage.

The 3D printed tissues may be used to study, e.g., tissue damage caused by pathogens and therapeutic compounds. For example, the 3D printed vascular tissues may be used for high content screening of small molecule libraries to identify molecules that treat or inhibit vascular dysfunctions caused by infection by hemorrhagic viruses, e.g., DENV. 3D printed vascular tissues may be printed on or provided in, e.g., a 96 well plate format, for high-throughput screening and imaging. High content imaging (HCl) may be used to assay gross changes in endothelial and perivascular cellular phenotypes during VHF pathogenesis. HCl analysis allows for the simultaneous measurement of multiple phenotypic features of the cell that may provide additional insights into the compound's mechanism of action or toxicity profile.

Antibodies against a given hemorrhagic virus may be used to identify the cells in a 3D printed tissue that are infected. For example, where the virus is DENV, antibodies 15F3-1, 3H5-1, 5D4-11, and 1H10-6 which are known in the art and are specific to DENV-1, -2, -3 and -4 serotypes, respectively, may be used and the nuclei and cell membranes of the infected cells in the 3D printed tissue may be stained and imaged and compared to that of uninfected controls. In screening assays, candidate compounds that inhibit or reverse changes in the cytoskeletal organization or vesicular compartments that are caused by a given hemorrhagic virus may be selected for further research and development of therapeutics and treatments against the given virus.

The 3D printed tissues can be used to study the molecular mechanisms that result in tissue damage, e.g., vascular dysregulation that is observed during DENV-induced shock in humans. Changes in the gene expression profile at RNA level (transcriptome) or protein level (proteomics) may be determined at the whole tissue or endothelial cellular level and correlated to the structural changes in the 3D printed tissue that is infected with the given virus using methods in the art.

### Kits

Kits are contemplated herein. In some embodiments, the kits comprise 3D printed tissue and/or a bioink for printing 3D tissue as disclosed herein. In some embodiments, the kits further comprise one or more reagents, e.g., detectable labels, blocking buffers, assay buffers, diluents, wash solutions, etc., for detecting and imaging cells of a tissue sample that are infected with a hemorrhagic virus. In some embodiments, the kits comprise cell culture media and/or one or more growth factors. In some embodiments, the kits comprise additional components such as interpretive information, control samples, reference levels, and standards.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

The following examples are intended to illustrate but not to limit the invention.

### ADDITIONAL EMBODIMENTS

Embodiment 1: A bioink composition comprising or consisting essentially of aortic smooth muscle cells and microvascular endothelial cells in an extracellular matrix solution which is a mixture of a gelatin solution, a basement membrane extract solution, and an ECL cell attachment matrix solution.

Embodiment 2: The bioink composition of Embodiment 1, wherein the gelatin solution is type A porcine skin gelatin at a concentration of 60 mg/mL; the basement membrane extract solution has a protein concentration of 15 mg/mL; the ECL cell attachment matrix solution has a protein concentration of 1 mg/mL; and wherein the extracellular matrix solution comprises the gelatin solution, the basement membrane extract solution, and the ECL cell attachment matrix solution in a ratio of 1:0.5:0.5 v/v.

Embodiment 3: The bioink composition of Embodiment 1 or Embodiment 2, wherein the concentration of the aortic smooth muscle cells is about 0.5×10⁶ to about 1.5×10⁶ cells/mL, preferably about 1×10⁶ cells/mL, of the extracellular matrix solution and the concentration of the microvascular endothelial cells is about 0.5×10⁶ to about 1.5×10⁶ cells/mL, preferably about 1×10⁶ cells/mL, of the extracellular matrix solution.

Embodiment 4: The bioink composition according to any one of Embodiments 1-3, wherein the basement membrane extract solution is lactose dehydrogenase elevating virus (LDEV) free.

Embodiment 5: The bioink composition according to any one of Embodiments 1 -4, wherein the basement membrane extract solution is LDEV free Gibco™ Geltrex™ available from Thermo Fisher Scientific.

Embodiment 6: A method of making a 3D printed tissue, which comprises using a bioprinter having a 22 gauge nozzle, 10-12 kPa printing pressure, a 3 mm/s travel speed, and a 12° C. printbed temperature to print the bioink composition according to any one of Embodiments 1-5 in a grid infill pattern at a 20% grid density.

Embodiment 7: The method according to Embodiment 6, which further comprises culturing the 3D printed tissue in a cell culture medium for 5 days at 37° C. Embodiment 8: A 3D printed tissue made by the method according to Embodiment 6 or Embodiment 7.

Embodiment 9: A kit comprising the bioink composition according to any one of Embodiments 1-5 and/or the 3D printed tissue according to Embodiment 8 packaged together with one or more reagents (e.g., buffers, growth media, cell culture media, detectable labels, etc.).

Embodiment 10: A method of assaying whether an agent likely changes the structure of vascular tissue, which comprises contacting the agent with a 3D printed tissue according to Embodiment 8 and then identifying any change in a cell structure in the 3D printed tissue, wherein a change in the cell structure in the 3D printed tissue indicates that the agent likely changes the structure of vascular tissue.

Embodiment 11: The method according to Embodiment 10, wherein the change in the cell structure is identified by comparing the cell structure in the 3D printed tissue which was contacted with the agent with a control.

Embodiment 12: The method according to Embodiment 11, wherein the control is a negative control (e.g., a 3D printed tissue that has not been contacted with the agent), a positive control (e.g., a 3D printed tissue that has been contacted with a given agent that is known to change the structure of vascular tissue), or a reference value (e.g., number of αSMA-positive cells present in a negative 3D printed control sample).

Embodiment 13: A method of assaying whether a test agent likely inhibits or treats vascular tissue damage, which comprises contacting the 3D printed tissue according to Embodiment 8 with (a) the test agent, and (b) an agent that is known to cause vascular tissue damage; and then identifying any change in a cell structure in the 3D printed tissue compared to a control, wherein the absence of a change in the cell structure in the 3D printed tissue indicates that the test agent likely inhibits or treats vascular tissue damage.

Embodiment 14: The method according to Embodiment 13, wherein the test agent is contacted with the 3D printed tissue before, concurrently with, or after contact with the agent.

Embodiment 15: The method according to any one of Embodiments 10-14, wherein the agent is a virus, preferably a virus that causes viral hemorrhagic fever.

Embodiment 16: The method according to any one of Embodiments 10-15, wherein the test agent is Zanamivir, Cathepsin-L, or Rock Inhibitor Y-27632.

### EXAMPLES

A 3D bioprinted multi-layer blood vessel wall model was constructed using a bioink consisting of human aortic smooth muscle cells and human microvascular lung endothelial cells suspended in a protein solution of gelatin, GELTREX basement membrane extract, entactin, collagen and laminin and “printed” using a CELLINK BIOX6 bioprinter. The specific cell markers of the printed construct were imaged and quantified using the Harmony 5.1 software of the Opera Phenix High Content Imager.

### Example 1: 3D Bioprinting of Vascular Tissue

3D bioprinted vascular tissue was made using human aortic smooth muscle cells (SMCs) and microvascular vascular endothelial cells (MVLECs) using a CELLINK BIOX6 printer and the following materials and protocol:

### Materials

CELLINK BIOX6 bioprinter
Gelatin from porcine skin, Type A (cat #G1890-1 KG, Sigma, St. Louis, MO) solution (60 mg/mL)
GELTREX, LDEV-free, undiluted (Cat #A14133-02, Gibco, Grand Island, NY)
Entactin/collagen IV/laminin (ECL) cell attachment matrix, undiluted (Cat #08-110, Millipore, Temecula, CA)·
PBS 1× (Corning, Manassas, VA)
Human Aortic Smooth Muscle Cells (Cat #CC-2571, Lonza, Walkersville, MD)
Microvascular Endothelial Cells (Cat #2527, Lonza, Walkersville, MD)
Smooth Muscle Cell Growth Medium with SmGM®-2 Bulletkit (Cat #CC-3182, Lonza, Walkersville, MD)
Microvascular Endothelial Cell Growth Medium (Cat #111-500, Cell Applications, Weathers Place, San Diego)

### Protocol

The BioInk comprises an extracellular matrix solution (Gelatin (60 mg/mL), GELTREX, and ECL matrix in 1:0.5:0.5 v/v, respectively) and cells. The preparation was performed aseptically and in a biosafety cabinet. GELTREX and ECL solutions were thawed on ice for about 30 minutes until the solution was liquid. The Gelatin (60 mg/mL) solution was heated via microwave until it melted. Volumes of Gelatin, GELTREX, and ECL solutions were mix together at a ratio of 1:0.5:0.5, respectively, to formulate the extracellular matrix solution. SMCs and MVECs were mixed with the extracellular matrix solution in amounts of about 1×10⁶ SMCs and about 1×10⁶ MVECs per 1 ml of the extracellular matrix solution to give the bioink. However, the amount of cells mixed with the extracellular matrix solution may be modified as desired. The bioink was loaded in a Bioink cartridge and then chilled at about 2-8° C. for about one hour.

After sterilizing the bioprinting chamber, the printbed temperature was set to 12° C. Then the Bioink cartridge was equipped with a 22-G printing needle and inserted in the printhead. After priming the printing needle, the tissue was printed using the bioprinter software, DNASTUDIO4, and programed to print a 5×5×1 simple shape having a 0.41 mm layer height, and a 66% first layer height via a pneumatic 3 ml print type, a 22-gauge nozzle type, a grid infill pattern, a 20% infill density, a 10 kPa printing pressure, a 3 mm/s travel speed, and a 12° C. printbed temperature.

Following printing, the 3D printed tissues were matured by culturing in culture media comprising Smooth Muscle Cell Growth Medium (Lonza, with SmGM®-2 Bulletkit) and Microvascular Endothelial Cell Growth Medium (Cell Applications, cat #11-500) at a 1:1 ratio for about 5 days at 37° C. (It was found that the 3D printed tissues remain intact after printing for at least 2 weeks by changing the culture media every other day.) After maturation, the 3D printed tissues were fixed with 10% formalin, and stained using VE-cadherin (endothelial cell marker), and smooth muscle actin (smooth muscle cell marker). Then, the 3D printed tissues were imaged using an Opera Phenix high content imager and image analysis was performed using Harmony 5.1 software. Total nuclei numbers and nuclei numbers of αSMA-positive cells were used as indicators of tissue viability, as well the amount of vascular tissue damage caused by hemorrhagic viral infection, e.g., infection by DENV.

1 ml of bioink results in about 5 tissue constructs, each construct having dimensions of 1 cm×1 cm×0.25 cm, i.e., a total volume of about 1.25 cm³ of printed tissue. Thus, after printing and before maturation, the 3D printed tissues comprise about 1×10⁶ SMCs and about 1×10⁶ MVECs per about 1.25 cm³ of printed tissue. That is, after printing and before maturation, the 3D printed tissues comprise about 8×10⁵ SMCs and about 8×10⁵ MVECs per about 1 cm³ of printed tissue. Human aortic SMCs have a cell proliferation rate, i.e., doubling time, of about 70-85 hours and MVECs have a doubling time of about 24-48 hours. Thus, the amount of the SMCs and MVECs in the printed tissues will presumably increase with time after printing, e.g., during the maturation phase before use.

### Example 2: Hemorrhagic Viral Infection and Staining

After five days of maturation, the 3D printed tissues were infected with Dengue virus (DENV) strain 2 (as a representative hemorrhagic virus) at 0.5 MOI for 72 hours in a registered BSL-2 lab for DENV infection.

Treatment using 100 μM Zanamivir, 20 μM Cathepsin-L Inhibitor, or 10 μM ROCK Inhibitor during the 72-hour infection alleviated DENV-driven cell loss, while Zanamivir and ROCK Inhibitor alleviated αSMA+ cell loss. The indicated amount of the given drug was added to the 3D printed tissues 2 hours prior to viral challenge.

Following infection, DENV was inactivated with 10% neutral buffered formalin (Fisher Scientific, Pittsburgh, PA) for three days. The formalin fixed 3D printed tissues were washed three times with phosphate-buffered saline (1×) (PBS). Then, the 3D printed tissues were blocked for one hour at room temperature with 3% bovine serum albumin (BSA) containing 0.3% Triton. Following blocking, the 3D printed tissues were probed overnight at 4° C. with primary antibodies which were diluted with 3% BSA. The primary antibodies were anti-DENV (D1-4G2, Novus Biologicals, Centennial, CO, 1:500 dilution), anti-smooth muscle actin (A5228, Sigma-Aldrich, St. Louis, MO, 1:100 dilution), and anti-VE-cadherin (D87F2, Cell Signaling, Danvers, MA, 1:100 dilution).

Then, the 3D printed tissues were washed three times with PBS, and incubated with secondary antibodies diluted with 3% BSA for one hour at room temperature. The secondary antibodies were Goat anti-mouse Alexa Fluor® 488 (Invitrogen, Rockford, IL, 1:1000), Goat anti-mouse Alexa Fluor® 568 (Invitrogen, Rockford, IL, 1:1000), Goat anti-rabbit Alexa Fluor® 568 (Invitrogen, Rockford, IL, 1:1000). The nuclei were stained with Hoechst (Invitrogen, Rockford, IL, 1:5000). Finally, the 3D printed tissues were washed three times with PBS and covered with additional PBS and sealed covers to prevent drying.

The stained 3D vascular tissues were imaged and analyzed using Opera Phenix High-Content Imager. A 20× Air magnification, Confocal Setting, and Z-stacks with 3.6 μm spacing to generate 3D renderings of the 3D printed tissues. Analysis and quantification were performed using Harmony Software version 5.1.

### Example 3: IPSC-Derived Endothelial and Smooth Muscle Cells

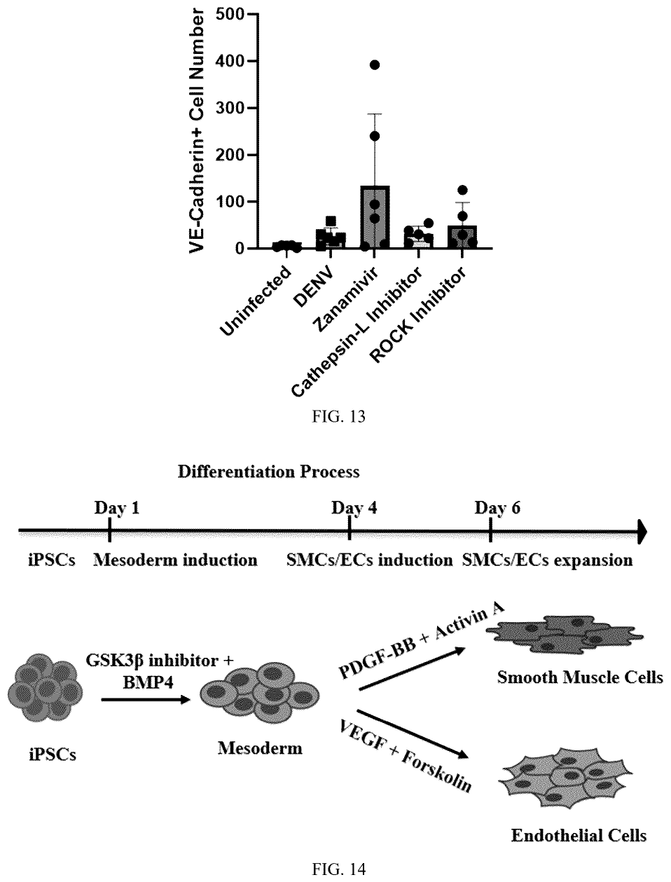

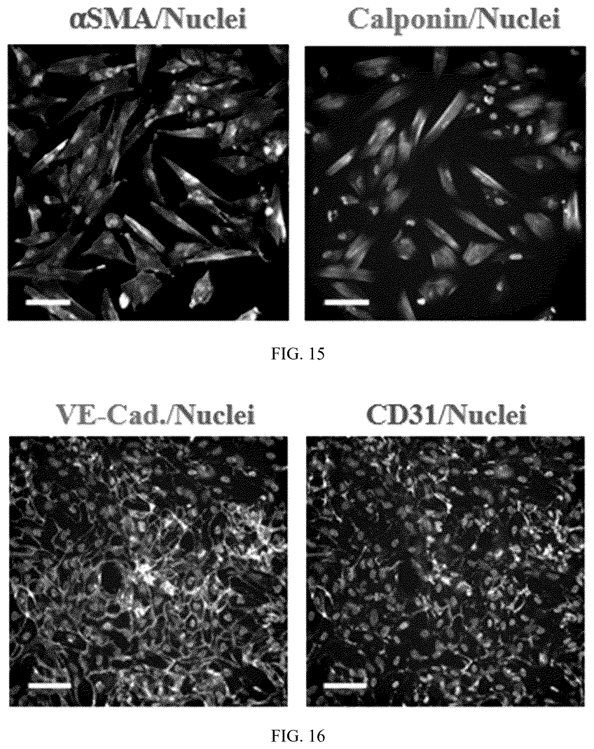

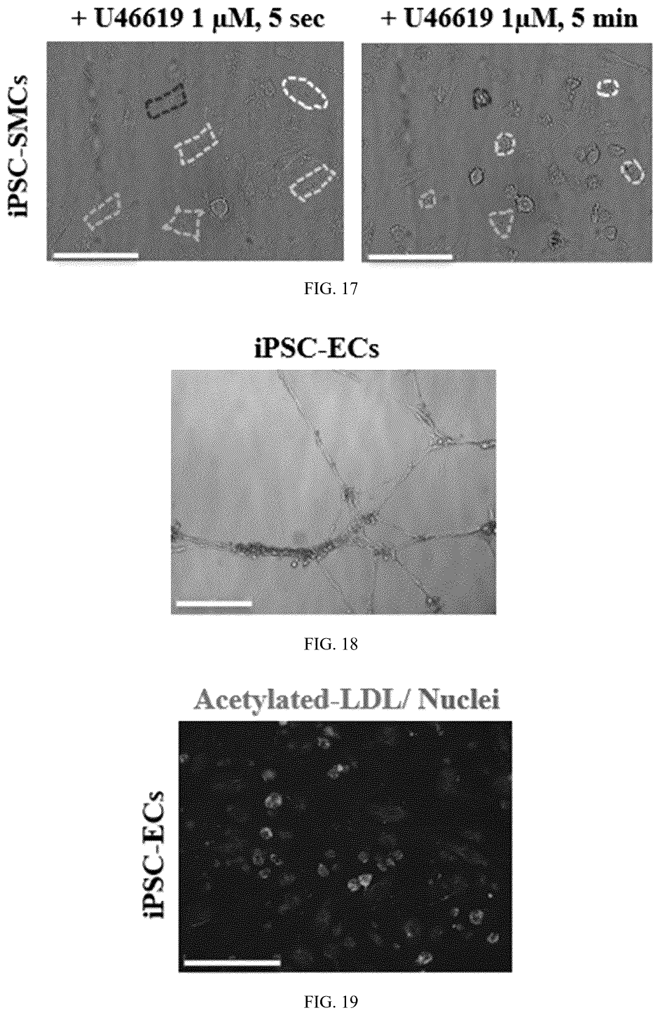

3D printed tissues comprising iPSC-derived smooth muscle and endothelial cells were generated and characterized. For human iPSC differentiation, the iPSC-lines were purchased from Takara Bio. iPSC-derived endothelial cells were differentiated using established protocols by Patsch, et al. and Gete, et al. with minor modifications. Briefly, iPSCs were committed to a mesodermal lineage through treatment with glycogen synthase kinase 3 beta (GSK3β) inhibitor and bone morphogenetic protein 4 (BMP4). Afterward, treatment with PGDF-BB and Activin A facilitated vascular SMC differentiation while vascular endothelial growth factor (VEGF) and forskolin facilitated endothelial cell differentiation (FIG. 14). Pure populations of (a) iPSC-SMCs that expressed α-smooth muscle actin (α-SMA) and calponin (FIG. 15) and (b) iPSC-ECs that expressed human vascular endothelial cadherin (hVE-Cadherin) and platelet endothelial cell adhesion molecule-1 (PECAM-1, also known as CD31) (FIG. 16) were obtained. iPSC-SMCs demonstrated contractile function and significant reduction in cell surface area following treatment of U46619, a vasoconstricting drug operating as a thromboxane A2 receptor agonist (FIG. 17). In addition, iPSC-ECs formed capillary-like microvascular networks in a tube formation assay (FIG. 18) and demonstrated uptake of Alexa 488-labeled acetylated Low-Density Lipoproteins (LDL) (FIG. 19).

These results demonstrate functional maturity of both cell types and suggest that the iPSC-SMCs and iPSC-ECs can faithfully use them for developing 3D bioprinting vascular tissue. Specifically, these results indicate that iPSC-derived vascular cells can be used in place of their primary counterparts because they express the same cell specific markers that are indicative of their functional properties. That is, whether cultured via conventional means or 3D printed in a matrix, iPSC-SMCs expressed α-smooth muscle actin (α-SMA) and calponin (specific smooth muscle cell markers), iPSC-ECs expressed human vascular endothelial cadherin (hVE-Cadherin), and platelet endothelial cell adhesion molecule-1 (PECAM-1, aka CD31) (specific endothelial cell markers). In addition, iPSC-SMCs demonstrated contractile function and showing significant reduction in cell surface area following treatment of U46619, a vasoconstricting drug operating as a thromboxane A2 receptor agonist. Moreover, iPSC-ECs formed capillary-like microvascular networks in a tube formation assay and demonstrated uptake of Alexa 488-labeled acetylated Low-Density Lipoproteins (LDL) (functional properties of endothelial cell). These results demonstrate functional maturity of both cell types as their primary counterparts and suggest that the iPSC-SMCs and iPSC-ECs can faithfully use them for developing 3D bioprinting vascular tissue.

N2B27 Medium: 500 ml DMEM/F12 medium (Life Technologies, #11320-033)+500 ml neurobasal medium (Life Technologies, #21103049), +20 ml Supplement B27 Minus Vitamin A (1.94%) (Life Technologies, #12587010)+10 ml N2 Supplement (0.97%) (100×, Life Technologies, #17502048)+1 ml β-Mercaptoethanol (0.097%) (50 mM, Life Technologies, #21985023), sterile filtration 0.22 μm.
Days 4/5 Medium: 500 ml StemPro®-34 medium with nutrient supplement (Life Technologies, #10639-011)+5 ml Pen/Strep (1:100), +5 ml GLUTAMAX (1:100) (Life Technologies, #35050038), +StemPro®-34 Supplement (Life Technologies, #10639-011).
EGM Medium: EGM-2 endothelial cell growth medium 2 with all bullet kit (Lonza, #CC-3162) added.
SMCG Medium: SmGM®-2 smooth muscle cell growth medium with all bullet kit (Loza, #CC-3182) added.

Maintenance of human induced pluripotent stem cells (hiPSCs): Human pluripotent stem cells (hiPSCs) purchased from Takarabio were routinely cultured on Cellartis® DEF-CS 500 COAT-1 (Takarabio, #Y30012) coated flasks in Cellartis® DEF-CS™ 500 Basal Medium (Takarabio, #Y30017) with growth factors. Cultures were passaged every 3-5 days using ACCUTASE (STEMCELL Technologies, #7920).

Day 0: Plating hiPSCs: Two T75 flasks were coated with growth factor reduced Matrigel® (BD/Fisher, #356230) by thawing on ice and diluting by 1:25. Flasks were incubated at room temperature for 1 hour. Matrices were aspirated, flasks were washed once with PBS and then 20 ml pre-warmed Cellartis® DEF-CS™ 500 Basal Medium (Takarabio, #Y30017) with all the growth factors was added to each flask. The hiPSCs were seeded at 40,000 hiPSCs per cm² and incubated at 37° C., 5% CO₂.

Day 1: Lateral mesoderm induction: Media in the flasks were replaced with 50 ml pre-warmed N2B27 Medium supplemented with 8 μM CHIR-99021 (Cayman, #13122)+25 ng/ml BMP4 (Peprotech, #120-05).

Endothelial cell induction and expansion: Day 4: The medium in the first flask was replaced with 50 ml of Days 4/5 medium supplemented with 200 ng/ml VEGF165 (Peprotech, #100-20) and 2 μM forskolin (Abcam, #ab120058). The medium was again replaced with the same on Day 5. Day 6: The medium was aspirated from the flask. The flask was then washed with 10 ml pre-warmed PBS (Ca²⁺ and Mg²⁺ free). 2 ml pre-warmed ACCUTASE (STEMCELL Technologies, #7920) was added and then the flask was incubated for 2-5 min at 37° C. Then cellular detachment was checked under a microscope. 5 ml of pre-warmed EGM Medium was added. Cells were transferred to a 15 ml Falcon tube and centrifuged at 1000 rpm (210 g) for 5 min. After discarding the supernatant, the cell pellet was resuspended in EGM Medium. The resuspended iPSC-derived endothelial cells (iPSC-ECs) were seeded at 30,000 per cm² on a fibronectin coated flask and incubated at 37° C., 5% CO₂. Cells were split when reaching 80% confluency.

Vascular smooth muscle cells induction and expansion: Day 4: The medium in the second flask was replaced with 50 ml N2B27 Medium supplemented with 10 ng/ml PDGF-BB (Peprotech, #100-14B) and 2 ng/ml Activin A (Peprotech, #120-14E). The medium was again replaced with the same on Day 5. Day 6: The medium was aspirated from the flask. The flask was then washed with 10 ml pre-warmed PBS (Ca²⁺ and Mg²⁺ free). 2 ml pre-warmed ACCUTASE (STEMCELL Technologies, #7920) was added and then the flask was incubated for 2-4 min at 37° C. Then cellular detachment was checked under a microscope. 5 ml prewarmed SMCG Medium was added. Cells were transferred to a 15 ml Falcon tube and centrifuged at 1000 rpm (210 g) for 5 min. After discarding the supernatant, the cell pellet was resuspended in SMCG Medium. The resuspended iPSC-derived smooth muscle cells (iPSC-SMCs) were seeded at 30,000 per cm² on a fibronectin coated flask and incubated at 37° C., 5% CO₂. Cells were split when reaching 80% confluency.

Characterization of iPSC-SMC and iPSC-ECs: For immunofluorescent staining, 4000 cells were seeded on 96 well plates. The following day, cells were washed once with PBS and fixed in 10% formalin for 30 min. Cells were then blocked with 3% BSA with (0.1 Triton) for 1 hour. iPSC-ECs were incubated with Vascular endothelial cadherin (D87F2) (Cell Signaling, #2500S, 1:100) and CD31 (Cell Signaling, #3228S, 1:100) overnight at 4° C. iPSC-SMCs were incubated with anti-smooth muscle actin (Milipore, #A5228, 1:100) and Calponin-1 (D8L2T) (Cell Signaling, #17819S, 1:100) overnight at 4° C. After three washes with PBS, cells were incubated in 3% BSA containing secondary antibodies and Hoechst (Invitrogen). Secondary antibodies used for immunocytochemistry were Alexa Fluor® 488 donkey anti-rabbit and Alexa Fluor® 647 donkey anti-mouse IgG (Invitrogen, 1:1000 dilution). Cells were washed three times with PBS, and images were acquired with Opera Phenix High Content Imager.

Tube Formation Assay: 75 μl of gel matrix (Corning® Matrigel® Basement Membrane Matrix Growth Factor Reduced, BD/Fisher, #356230) was aliquoted to 96-well plates and incubated for 30 min at 37° C. to allow the gel to solidify. 10,000 iPSC-ECs were seeded onto the gel matrix and cultured for 18 hours at 37° C., 5% CO₂, and images were acquired.

### REFERENCES

The following references are herein incorporated by reference in their entirety with the exception that, should the scope and meaning of a term conflict with a definition explicitly set forth herein, the definition explicitly set forth herein controls:
Patsch, et al. (2015) Generation of vascular endothelial and smooth muscle cells from human pluripotent stem cells. Nat Cell Biol. 17(8):994-1003.
Gete, et al. (2021) Mechanisms of angiogenic incompetence in Hutchinson-Gilford progeria via downregulation of endothelial NOS. Aging Cell. 20(7):e13388.

All scientific and technical terms used in this application have meanings commonly used in the art unless otherwise specified.

As used herein, a “viral hemorrhagic fever (VHF)” refers to an infection by a hemorrhagic virus and the illness caused thereby. VHF is caused by viruses belonging to 3 families (Arenaviridae, Filoviridae, Flaviviridae) and 1 order (Bunyavirales) of enveloped RNA viruses. Arenaviridae (arenaviruses) include Chapare (CHAPV), Guanarito (GTOV), Junin (JUNV), Lassa (LASV), and Lujo viruses (LUJV); lymphocytic choriomeningitis virus (LCMV); and Machupo (MACV) and Sabia (SBAV) viruses. Viruses belonging to the order Bunyavirales include the Arenaviridae family viruses, Crimean-Congo hemorrhagic fever (CCHF) virus (family Nairoviridae), hantaviruses (family Hantaviridae), and Rift Valley fever (RVF) virus (family Phenuiviridae). Filoviridae (filoviruses) include Ebola (EBOV), Marburg (MARV), and Reston (RESTV) viruses. Flaviviridae (flaviviruses) include Alkhurma (ALKV), Kyasanur Forest disease (KFDV), Omsk hemorrhagic fever (OHFV), dengue (DENV), and yellow fever (YFV) viruses. Thus, in some embodiments, the hemorrhagic virus belongs to the Arenaviridae, Filoviridae, or Flaviviridae family of viruses or the Bunyavirales order of viruses. In some embodiments the hemorrhagic virus is a mammalian hantavirus, e.g, Hantaan orthohantavirus (HTNV) and Sin Nombre orthohantavirus (SNV). In some embodiments, the hemorrhagic virus is DENV, CHAPV, GTOV, JUNV, LASV, LUJV, LCMV, MACV, SBAV, CCHF, RVF, EBOV, MARV, RESTV, ALKV, KFDV, OHFV, YFV, HTNV, or SNV.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”. As used herein, the phrase “consists essentially of” in the context of a given ingredient in a composition, means that the composition may include additional ingredients so long as the additional ingredients do not adversely impact the activity, e.g., biological or pharmaceutical function, of the given ingredient. In the context of the bioinks herein, “consists essentially of” means that the bioinks may comprise additional ingredients (e.g., buffers, growth factors, etc.) so long as the additional do not affect the structure and function of the aortic smooth muscle cells and microvascular endothelial cells therein.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A bioink composition comprising aortic smooth muscle cells and microvascular endothelial cells in an extracellular matrix solution which is a mixture of a gelatin solution, a basement membrane extract solution, and an ECL cell attachment matrix solution.

2. The bioink composition of claim 1, wherein the gelatin solution is type A porcine skin gelatin at a concentration of 60 mg/mL; the basement membrane extract solution has a protein concentration of 15 mg/mL; the ECL cell attachment matrix solution has a protein concentration of 1 mg/mL; and wherein the extracellular matrix solution comprises the gelatin solution, the basement membrane extract solution, and the ECL cell attachment matrix solution in a ratio of 1:0.5:0.5 v/v.

3. The bioink composition of claim 1, wherein the concentration of the aortic smooth muscle cells is about 0.5×10⁶ to about 1.5×10⁶ cells/mL, preferably about 1×10⁶ cells/mL, of the extracellular matrix solution and the concentration of the microvascular endothelial cells is about 0.5×10⁶ to about 1.5×10⁶ cells/mL, preferably about 1×10⁶ cells/mL, of the extracellular matrix solution.

4. The bioink composition according to claim 1, wherein the basement membrane extract solution is lactose dehydrogenase elevating virus (LDEV) free.

5. The bioink composition according to claim 1, wherein the basement membrane extract solution is LDEV free Gibco™ Geltrex™ available from Thermo Fisher Scientific.

6. A method of making a 3D printed tissue, which comprises using a bioprinter having a 22 gauge nozzle, 10-12 kPa printing pressure, a 3 mm/s travel speed, and a 12° C. printbed temperature to print the bioink composition according to claim 1 in a grid infill pattern at a 20% grid density.

7. The method according to claim 6, which further comprises culturing the 3D printed tissue in a cell culture medium for 5 days at 37° C.

8. A 3D printed tissue made by the method according to claim 6.

9. A kit comprising the bioink composition according to claim 1 packaged together with one or more reagents (e.g., buffers, growth media, cell culture media, detectable labels, etc.).

10. A kit comprising the 3D printed tissue according to claim 8 packaged together with one or more reagents (e.g., buffers, growth media, cell culture media, detectable labels, etc.).

11. A method of assaying whether an agent likely changes the structure of vascular tissue, which comprises contacting the agent with a 3D printed tissue according to claim 8 and then identifying any change in a cell structure in the 3D printed tissue, wherein a change in the cell structure in the 3D printed tissue indicates that the agent likely changes the structure of vascular tissue.

12. The method according to claim 11, wherein the change in the cell structure is identified by comparing the cell structure in the 3D printed tissue which was contacted with the agent with a control.

13. The method according to claim 12, wherein the control is a negative control (e.g., a 3D printed tissue that has not been contacted with the agent), a positive control (e.g., a 3D printed tissue that has been contacted with a given agent that is known to change the structure of vascular tissue), or a reference value (e.g., number of αSMA-positive cells present in a negative 3D printed control sample).

14. A method of assaying whether a test agent likely inhibits or treats vascular tissue damage, which comprises contacting the 3D printed tissue according to claim 8 with (a) the test agent, and (b) an agent that is known to cause vascular tissue damage; and then identifying any change in a cell structure in the 3D printed tissue compared to a control, wherein the absence of a change in the cell structure in the 3D printed tissue indicates that the test agent likely inhibits or treats vascular tissue damage.

15. The method according to claim 14, wherein the test agent is contacted with the 3D printed tissue before, concurrently with, or after contact with the agent.

16. The method according to claim 11, wherein the agent is a virus, preferably a virus that causes viral hemorrhagic fever.

17. The method according to claim 11, wherein the test agent is Zanamivir, Cathepsin-L, or Rock Inhibitor Y-27632.
